(19) United States
(12) Patent Application Publication
(10) Pub. No.: US 2007/0004987 A1
(43) Pub. Date: Jan. 4, 2007

(54) Device for acquiring electric signals coming from the human body and more particularly from the heart

(75) Inventors: David Oury, Sainte Foy Les Lyon (FR); Maurice Oury, Sainte Foy Les Lyon (FR)

Correspondence Address: YOUNG & THOMPSON, 745 SOUTH 23RD STREET, 2ND FLOOR, ARLINGTON, VA, 22202, US

(73) Assignee: C 2 C SA, SAINTE FOY LES LYON (FR)

(21) Appl. No.: 11/518,227

(22) Filed: Sep. 11, 2006

Related U.S. Application Data
(62) Division of application No. 10/333,411, filed on Jan. 21, 2003, now abandoned.

(30) Foreign Application Priority Data
Jul. 19, 2000 (FR) 00/09483

(51) Int. Cl. A61B 5/04

(52) U.S. Cl. 600/509

(57) ABSTRACT

A device for acquiring electric signals of the human body from a patient's heart includes at least two independent connection elements that each has supports that are provided each with electrodes. The electrodes are attached to an electric cable with parallel strands, which is connected to an input of an acquisition card that receives and transmits, free of electromagnetic interference, electric signals derived from the electrodes to a central unit for data processing, analyzing and editing.

### BACKGROUND OF THE INVENTION

The present invention relates to a device for acquiring electric signals coming from the human body, and more particularly for acquiring electric signals coming from the heart in order to produce electrocardiograms.

The electrocardiograph is the basic tool for studying cardiovascular pathologies. There are many types of electrocardiograph producing studies which are complete to a greater or lesser degree.

The complexity of an electrocardiograph is determined by the number of leads, since the larger the number of leads, the more plentiful the information coming from the heart. At present, there are electrocardiographs which have from one to twelve leads.

To produce an electrocardiogram with 12 leads, it is necessary to place on patient's body ten electrodes connected to the electrocardiograph by electric wires.

The ten electrodes are placed as follows:
six electrodes on the thorax,
four electrodes distributed over the extremities of the patient's body at the wrists and ankles.

The electric signals picked up by the electrodes can be used by the electrocardiograph, either directly on a paper medium, or via a computer having special display and calculation software.

To acquire electric signals, there are two types of electrodes: either bulb electrodes, or pregelled electrodes.

Bulb electrodes are suction pads entailing the application of a gel to the region of the patient's body receiving the electrode to allow good adhesion. The practitioner must place the electrodes one by one at the points on the thorax, the wrists and the ankles.

Each suction-pad electrode is connected by an electric wire to a specific input of the electrocardiograph depending on its position on the patient's body.

Pregelled electrodes are similar to those described in patents U.S. Pat. No. 4,393,584, U.S. Pat. No. 4,559,950, U.S. Pat. No. 4,082,087 and EP 0 284 943 in that they have a self-adhesive structure which is placed on the patient's body. Each electrode is then connected to an electric wire via either a crocodile clip or a press stud.

When the self-adhesive structure has several electrodes as in patent U.S. Pat. No. 4,082,087, the latter are directly connected to the electrocardiograph by electric wires incorporated in the structure.

It is found that the devices for acquiring electric signals described above have, between the position of the electrodes fastened to the patient's body and the electrocardiograph, a large surface electromagnetic field through which electromagnetic waves pass, interfering with the electrocardiograph trace.

It is these drawbacks that the acquisition device according to the present invention more particularly intends to overcome.

### SUMMARY OF THE INVENTION

The device for acquiring electric signals according to the present invention comprises at least two independent connection elements respectively having supports, each one provided with electrodes connected via a parallel-strands electric cable which is connected to an input of an acquisition card for receiving and transmitting, without electromagnetic interference, electric signals coming from the electrodes to a central processing unit for processing, analyzing and printing out data.

The device for acquiring electric signals according to the present invention comprises at least two independent connection elements respectively comprising:
two supports, each one provided with three electrodes connected via an electric cable which is connected to an input of the acquisition card;
two other supports, each one provided with two electrodes connected via an electric cable which is connected to an input of the acquisition card.

The device for acquiring electric signals according to the present invention comprises a first connection element which is fastened to the body of the patient at the thorax so that each support with three electrodes is placed in a vertical direction on either side of the vertical axis XX′ displaying the position of the sternum.

The device for acquiring electric signals according to the present invention comprises a second connection element which is fastened to the body of the patient below the heart and so that each support with two electrodes is oriented in a substantially curved direction running downward and to the right side of the patient.

The device for acquiring electric signals according to the present invention comprises a support which is made from a woven or nonwoven tape, provided with a self-adhesive face suitable for accommodating the electrodes, and which has a T-shaped profile delimited by an elongate part whose ends are rounded, and another rectangular part of larger surface area than the previous and which extends the latter on its side having the greatest length.

The device for acquiring electric signals according to the present invention comprises a self-adhesive face which is covered with a protective film made of paper or the like, having a profile similar to that of the support in order to cover all the surfaces of the parts.

The device for acquiring electric signals according to the present invention comprises a protective film which is cut in order to form a first detachable element, on the one hand, completely covering the surface of the elongate part of the support, and on the other hand, covering half of the surface of the rectangular part of said support, and a second detachable element which covers the other half of the remaining surface of the part of the support.

The device for acquiring electric signals according to the present invention comprises electrodes which each have a surface covered with gel for picking up electric signals and a surface for the electric connection of the cable.

The device for acquiring electric signals according to the present invention comprises electrodes which are aligned on the support so that each surface is stuck, after removing the first detachable element of the protective film, to the self-adhesive face of the elongate part, while each surface is stuck to the self-adhesive face corresponding to half the surface of the rectangular part.

The device for acquiring electric signals according to the present invention comprises electrodes which are positioned so that each connection surface covers part of the self-adhesive face of the rectangular part which is not protected by the second detachable element, so as to form, between the edge of the detachable element and the surfaces, an uncovered self-adhesive space for putting the electric cable in place.

The device for acquiring electric signals according to the present invention comprises a second detachable element of the protective film which is removed in order to uncover the other half of the self-adhesive face of the rectangular part in order to be folded around the cable and to be stuck to the other half so as to hold said cable on the support and to protect the connections of the electric wires on the connection surface of each electrode.

The device for acquiring electric signals according to the present invention comprises a first detachable element of the protective film which has a tab.

The device for acquiring electric signals according to the present invention comprises a rectangular part of the support which is folded around the cable and strengthened by self-adhesive tape made from a woven or nonwoven material.

The device for acquiring electric signals according to the present invention comprises self-adhesive tape which is positioned between the cable and the folded edge of the tab of the first detachable element of the protective film so as to fasten the two folded halves of the rectangular part over each other perfectly.

The device for acquiring electric signals according to the present invention comprises self-adhesive tape which is provided in order to accommodate, on one of its faces, and more particularly the face which is opposite the first detachable element, an advertising label.

The device for acquiring electric signals according to the present invention comprises an electric cable which is connected at one of its ends to a connector, for its connection to the input of the acquisition card.

### BRIEF DESCRIPTION OF THE DRAWINGS

The following description, with reference to the appended drawings given by way of nonlimiting examples, will make it easier to understand the invention, its features and the advantages that it is likely to afford:

FIG. 1 is a schematic view showing the position on the body of a patient of some of the electrodes of an acquisition device of the prior art and the area of the electromagnetic field induced in the loop formed by the leads.

FIG. 2 is a schematic view illustrating a trace obtained by the acquisition device of the prior art of FIG. 1.

FIG. 3 is a schematic view showing the position, identical to that of FIG. 1, of some of the electrodes of the acquisition device according to the present invention.

FIG. 4 is a schematic view showing a trace obtained by the acquisition device according to the present invention and according to FIG. 3.

FIG. 5 is a schematic view illustrating the placement on the body of an adult patient of all the electrodes of the acquisition device according to the present invention.

FIG. 6 is a schematic view illustrating the placement on the body of a young patient of all the electrodes of the acquisition device according to the present invention.

FIGS. 7 to 13 are views showing the structure of the support accommodating the electrodes, given that each support may accommodate either one electrode, or two electrodes, or three electrodes depending on the mode of use of the acquisition device according to the present invention.

### DESCRIPTION OF THE PREFERRED EMBODIMENTS

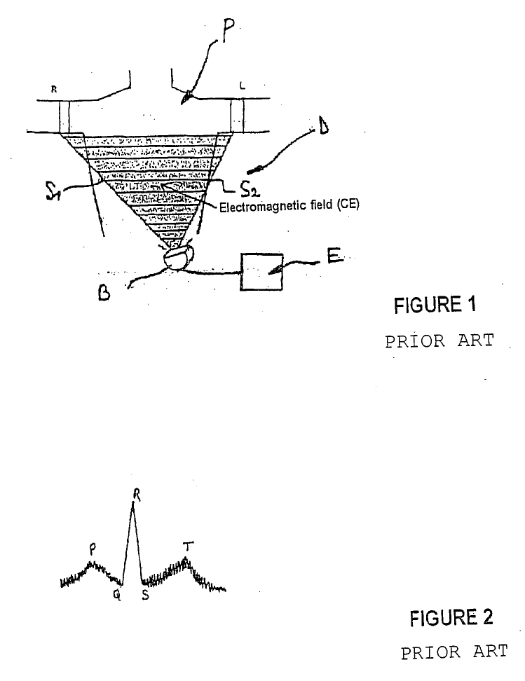

FIG. 1 shows the position on the body of an adult patient P of some of the electrodes and especially those referenced L and R of an acquisition device D of the prior art.

The electrodes L and R are respectively connected via a box B to an electrocardiograph E by a cable S1 and S2 to allow transmission of the electric signals.

It is found that the loop formed by the cables S1 and S2 of the electrodes L, R and the box B of the electrocardiograph E allows the formation of an electromagnetic field CE whose area varies according to the position of the electrodes on the body of the patient P and to the distance between the electrodes and the electrocardiograph.

Thus, the area of the electromagnetic field CE is induced in the loop formed by the cables S1, S2 of the electrodes L, R and the box B of the electrocardiograph E.

Furthermore, there is an electromagnetic field CE for each measurement made between two electrodes of the acquisition device D of the prior art which has a total of twelve leads, that is twelve electrodes. It is found that the greater the area of the electromagnetic field CE, the greater are the chances that electromagnetic waves coming from electric apparatus located in the environment close to the human body P will pass through this same field.

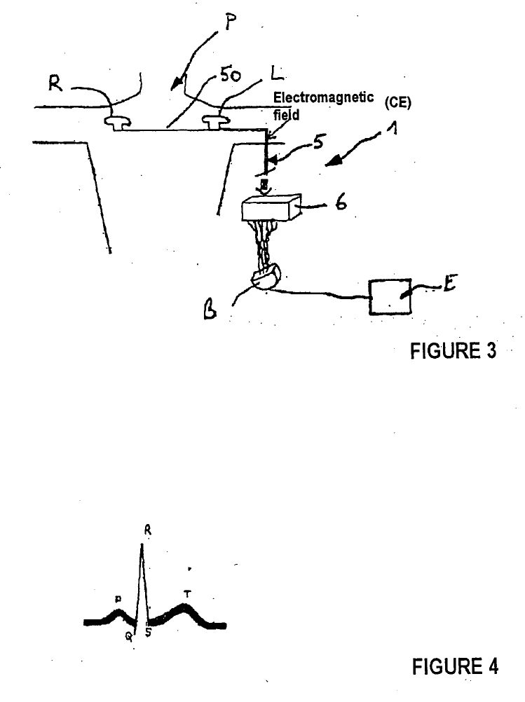

FIG. 2 shows the result of the trace coming from the electric signals captured by the electrodes L and R of the acquisition device D of the prior art.

It is found that the graphical representation of the trace has interference, that is to say, that it consists of series of irregular vertical lines of a varying size interfering with the sharpness and the linear regularity of the trace.

This graphical representation has interference from the electromagnetic waves which are close to the acquisition device D of the prior art.

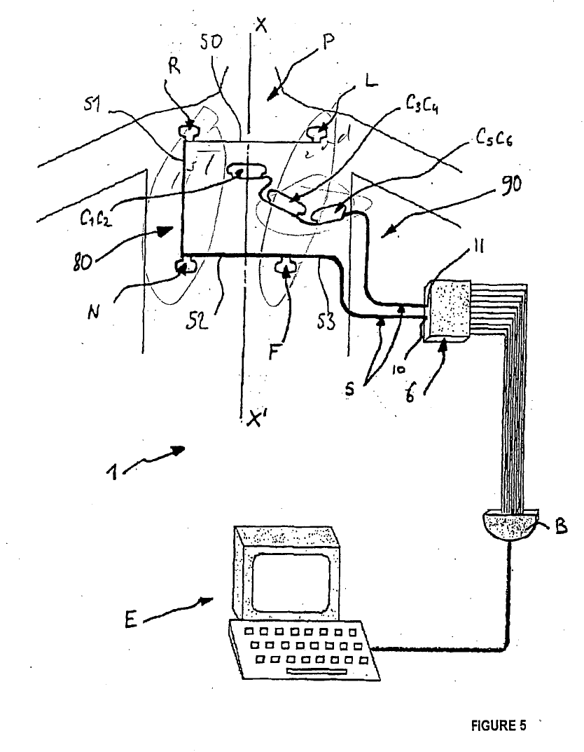

FIG. 3 shows some of the electrodes of a device 1 for acquiring electric signals according to the present invention and more particularly the electrodes L and R so as to be able to compare the results of the traces obtained from the two devices.

The electrodes L and R of the acquisition device 1 are stuck to the body of an adult patient P in order to produce an electrocardiogram.

The electrode L is connected to the electrode R by a strand 50 of a cable 5 enabling both electrodes to be connected to an acquisition card or adapter 6.

The card 6 can be connected either directly to a central processing unit consisting of a computer 7 provided with software for processing and printing-out data, or to the box B of an electrocardiograph E, known per se.

The cable 5 consists of parallel strands assembled against each other, the number of which varies according to the number of electrodes to be connected to the card 6.

As a result, the area of the electromagnetic field CE is limited to the distance between each parallel strand of the cable 5.

The construction of the cable 5 makes it possible to eliminate the electromagnetic field induced by the loop formed by the leads and to remove the electromagnetic interference coming from the electric apparatuses which are close to the acquisition device 1.

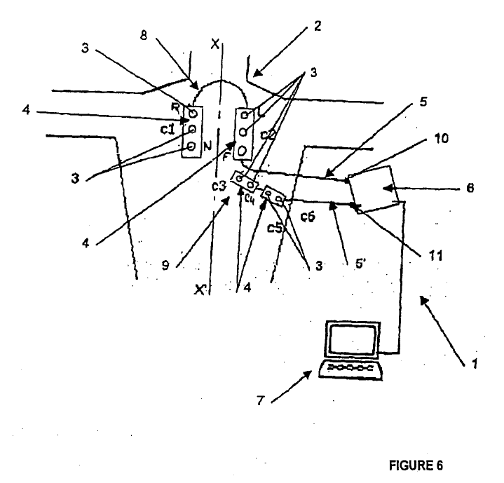

FIG. 4 shows the result of the trace coming from the electric signals captured by the electrodes L and R of the acquisition device 1 according to the present invention.

It is found that the graphical representation of this trace is purer than that shown in FIG. 2.

This is because of an overall decrease in noise on the curves, this noise being directly associated with the electromagnetic waves coming from the nearby electric apparatuses.

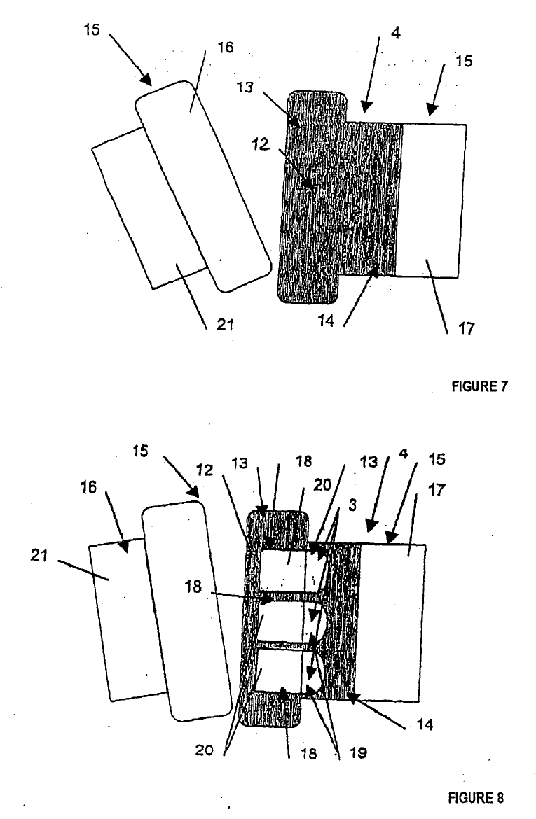

FIG. 5 shows the position, on the body of an adult patient P, of each electrode of the device 1 for acquiring electric signals according to the present invention.

The acquisition device 1 consists of two independent connection elements 80, 90 for picking up electric signals, which are placed in different regions of the body of the patient P, and each connected to a different input of the acquisition card 6.

The first connection element 80 consists of electrodes referenced L, R, N and F. The references L, R, N and F are the terms commonly used for the peripheral electrodes of an electrocardiogram to identify the position of each electrode on the body of the patient P.

The first connection element 80 comprises four supports 4, each one provided with an electrode 3, which are connected by a cable 5 to the input 10 of the acquisition card 6.

In this case, the cable 5 consists of four parallel and independent strands 50, 51, 52, 53 connecting each electrode 3 to the input 10 of the card 6.

The first connection element 80 is fastened to the body of the adult patient P so that each support 4 is placed in such a way as to form the corners of a square substantially centered on the vertical axis XX′ displaying the position of the sternum.

The second connection element 90 consists of the electrodes referenced C1C2, C3C4 and C5C6.

The second connection element 90 comprises three supports 4 each one provided with two electrodes 3 which are connected by another cable 5 to the input 11 of the acquisition and 6.

In this case, the cable 5 consists of six parallel and independent strands 54 to 59 connecting each electrode 3 to the input 11 of the card 6.

The second connection element 90 is fastened to the body of the patient P below the heart and so that each support 4 with two electrodes 3 is oriented in a substantially curved direction running downward and to the right side of the patient.

The acquisition card or adapter 6 is connected, in this example, to an electrocardiograph E consisting of a central processing unit 7 via a box B.

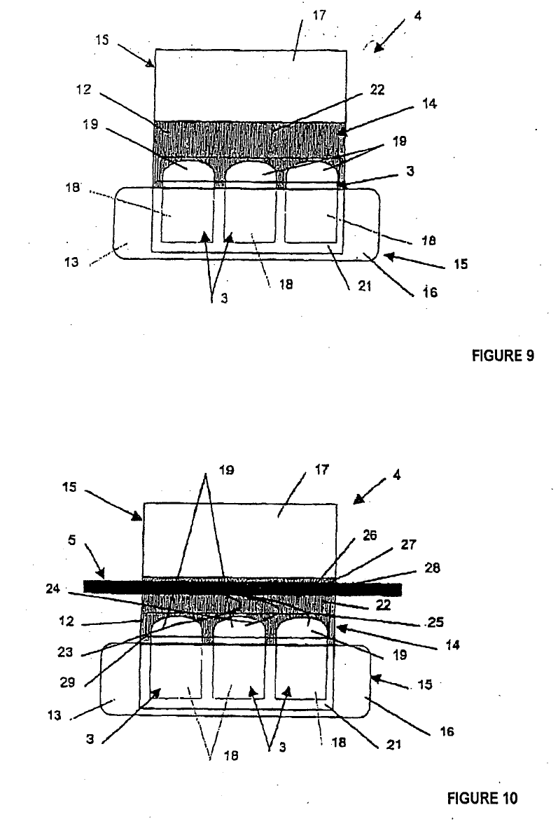

FIG. 6 shows a device 1 for acquiring electric signals which is fastened and stuck to the body of a young patient 2 for producing an electrocardiogram.

The acquisition device 1 comprises electrodes 3 stuck to supports 4, so that each electrode is connected by electric cables 5, 5′ to a card 6 for acquiring the electric signals.

The acquisition card 6 is connected to a central processing unit forming a computer 7, in this case provided with software for processing and printing out data coming from the card 6.

The acquisition card is of the modem type having an integrated microprocessor which automatically manages, on the one hand, the connections of the cables 5, 5′, and on the other hand, the acquisition of the electric signals coming from the electrodes 3 so as to save them to memory if need be.

The processing software provided in the computer 7 has basic functions, advanced functions and a patient file.

The highly intuitive basic functions considerably simplify the practitioner's work when analyzing the traces. Functions for comparing each track can be found on the computer screen, one function allowing the full-screen single-window reading of the track, a zoom function, a scrollbar function, etc.

The advanced functions have all the functionalities of a twelve-lead electrocardiogram (EKG), such as automatic P-QRS-TPR calculations, but above all they make it possible to carry out normality studies bearing on the following criteria:
cardiac morphology,
cardiac rhythm disorders,
cardiac contractility.

These studies are made possible by virtue of the acquisition device 1, which is the subject of the present invention, with an electronic stethoscope as described and protected in patents FR 2 767 467 and FR 2 767 468.

The acquisition device 1 has two independent connection elements 8, 9 for picking up the electric signals, which are placed in different regions of the body of the patient 2, and each connected to a different input of the acquisition card 6.

The first connection element 8 consists of electrodes referenced L, R, N, F, C1 and C2. The references L, R, N and F are the terms commonly used for the peripheral electrodes, while the references C1 and C2 are the terms used for the pericordial electrodes of an electrocardiograph.

The first connection element 8 has two supports 4, each one provided with three electrodes 3 which are connected by a cable 5 to the input 10 of the acquisition card 6.

The first connection element 8 is fastened to the body of the patient 2 at the thorax so that each support 4 with three electrodes 3 is placed in a vertical direction on either side of the vertical axis XX′ displaying the position of the sternum.

The second connection element 9 consists of the electrodes referenced C3, C4, C5 and C6. The references C3, C4, C5 and C6 are the terms used for the pericordial electrodes of an electrocardiograph.

The second connection element 9 has two supports 4, each one provided with two electrodes 3 which are connected by another cable 5′ to another input 11 of the acquisition card 6.

The second connection element 9 is fastened to the body of the patient 2 below the heart and so that, each support 4 with two electrodes 3 is oriented in a substantially curved direction running downward and to the right side of the patient.

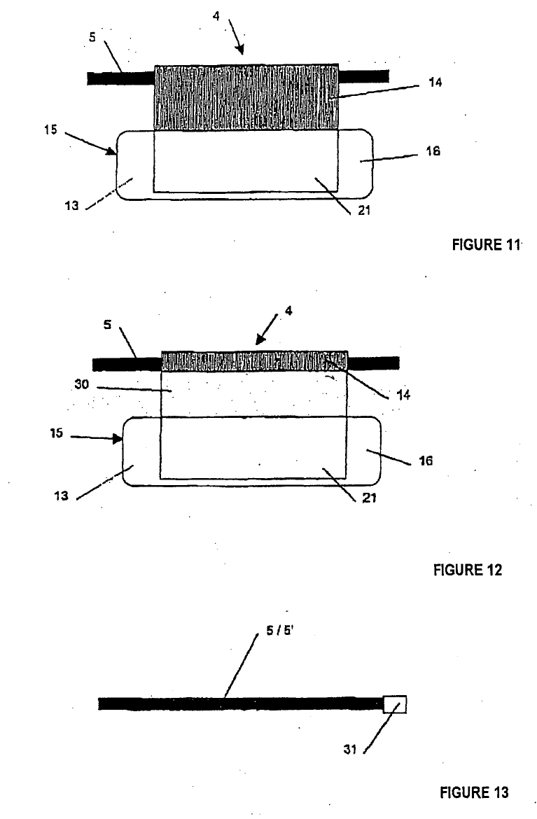

FIGS. 7 to 13 show an example of a support 4 provided for the connection elements 8, 9; 80, 90 of the acquisition device 1 according to the present invention.

The support 4, described by way of example in FIGS. 7 to 13, comprises three electrodes 3, given that the structures of the other supports are identical and that these supports may, depending on the connection elements, comprise either one, two or three electrodes depending on the conditions of use of the acquisition device 1 according to the present invention.

FIG. 7 shows a support 4 made from a woven or nonwoven tape, provided with a self-adhesive face 12 suitable for accommodating the electrodes 3.

The support 4 has a T-shaped profile or the like delimited by an elongate part 13 whose ends are rounded, and another rectangular part 14 having a area larger than that 13 and which extends the latter on its longest side.

The self-adhesive face 12 is covered with a protective film 15 made of paper or the like having a profile similar to that of the support 4 so as to cover all the surfaces of the parts 13 and 14.

The protective film 15 is cut in order to form a first detachable element 16, on the one hand, completely covering the surface of the elongate part 13 of the support 4, and, on the other hand, covering half the surface of the rectangular part 14 of the support 4.

The protective film 15 has a second detachable element 17 which covers the other half of the remaining surface of the part 14 of the support 4.

FIG. 8 shows the support 4, the first detachable element 16 of the protective film 15 of which has been removed in order, on the one hand, to completely uncover the self-adhesive face 12 of the elongate part 13 and, on the other hand, to uncover that half of the self-adhesive face 12 covering the rectangular part 14.

Removing the element 16 makes it possible to stick either one, two or three electrodes 3, depending on the embodiment, of either the first connection element 8, 80, or the second 9, 90 onto the self-adhesive face 12 of the support 4.

The electrodes 3 have a surface 18 for picking up electric signals and a surface 19 with a curved profile for connecting the corresponding strands of each electric cable 5, 5′.

The square-shaped surface 18 of each electrode 3 is covered with a gel 20 facilitating contact with the skin of the patient 2.

The electrodes 3 are aligned on the support 4 so that each square surface 18 is stuck to the self-adhesive face 12 of the elongate part 13, while the surface 19 with a curved profile is stuck in the uncovered region of the self-adhesive face 12 representing half the surface of the rectangular part 14.

FIG. 9 shows the support 4 fitted with the electrodes 3, the number of which varies according to the embodiment of the connection element 8, 80 or of the connection element 9, 90.

The detachable element 16 of the protective film 15 is prefolded in order to form a tab 21 before it is put in place on the self-adhesive face 12 of the elongate part 13 of the support 4, so as to protect only the surface 18 with a square profile of the electrodes 3.

The electrodes 3 are positioned so that each curved surface 19 covers part of the self-adhesive face 12 of the rectangular part 14 not protected by the detachable element 17, so as to form a self-adhesive free space 22 between the edge of the detachable element 17 and the curved surfaces 19.

FIG. 10 shows the support 4 fitted with the electrodes 3 connected to the parallel strands, for example, of the electric cable 5 which is stuck in the free space 22 of the rectangular part 14.

The electric cable 5 is partially stripped beforehand on one of its faces in order to uncover three electric wires or strands 23 to 25 out of a total of six, given that the other wires or strands 26 to 28 will enable the other support 4 forming, for example, the first connection element 8, to be connected.

The ends of the wires or strands 23 to 25 are stripped and positioned respectively on the curved surface 19 of each electrode 3 of the support 4.

A self-adhesive film 29 fastens the wires or strands 23 to 25 to the curved surface 19 of each electrode 3 in order to enable the connection and electric contact between each electrode 3 and its respective wire or strand.

FIGS. 11 and 12 show the support 4 fitted with the electrodes 3 connected to the electric cable 5 for producing, for example, the connection element 8.

The detachable element 17 of the protective film 15 is removed in order to uncover the other half of the self-adhesive face 12 of the rectangular part 14.

This half of the rectangular part 14 is folded around the cable 5 in order to be stuck to the other half so as to hold said cable on the support 4 and protect the connections of the electric wires or strands 23 to 26 on the curved surface 19 of each electrode 3.

This half of the rectangular part 14 is folded around the cable 5 so that its free edge is in immediate proximity to the elongate surface 13 protected by the detachable element 16, provided with its tab 21, of the protective film 15.

The rectangular part 14 of the support 4 folded around the cable 5 is reinforced by a self-adhesive tape 30 made from a woven or nonwoven material.

The self-adhesive tape 30 is positioned between the cable 5 and the folded edge of the tab 21 of the detachable element 16 of the protective film 15, so as to fasten the two folded halves of the rectangular part 14 perfectly over each other in order to prevent any opening of the latter.

The self-adhesive tape 30 is designed to accommodate, on one of its faces, and more particularly the face opposite the detachable element 16, an advertising label.

FIG. 13 shows the electric cable 5 which is connected at one of its ends to a connector 31 for its connection with the input 10 of the acquisition card 6.

The connectors 31 connected at one of the ends of the cables 5 and 5′ have different dimensions so as to prevent any connection error by the practitioner at the acquisition card 6.

The acquisition device 1 according to the present invention comprises, in each configuration, two connection elements 8, 9; 80, 90 enabling electric signals to be acquired from the body of the adult P or young 2 patient.

Putting the connection elements 8, 9 and 80, 90 on the body of the adult P or young 2 patient is very easy, since all that is needed is for the practitioner to remove the detachable element 16 of the protective film 15 by pulling on the tab 21 in order to uncover the self-adhesive face 12 corresponding to the elongate part 13 of the support 4.

The practitioner then applies the supports 4 of each connection element 8, 9 or 80, 90 depending on the patient and in a predetermined position, so that the surfaces 18 covered with gel 20 of each electrode 3 are in contact with the skin of the adult P or young 2 patient.

Finally, the practitioner connects the cables 5 and 5′ of the connection elements 8, 9 or 80, 90 to the corresponding inputs 10 and 11 of the acquisition card 6 so that the latter saves the electric signals coming from the heart to its memory.

The data recovered by the acquisition card 6 are transmitted either to the electrocardiograph E, or to the computer 7 which will process said data by means of the software so that the practitioner obtains the traces of the electrocardiogram from his adult P or young 2 patient.

It is found that the profile of the electrodes 3 may be different without however changing the subject of the invention. This is because each electrode must have a region for picking up electric signals which region may or may not be covered with gel, and an electric connection region.

It is noted that the cables 5 and 5′ may be produced as parallel wires or strands in the form of a ribbon or as printed or integrated circuits, without however changing the subject of the invention.

It is noted that the practitioner may supplement his examination using an electronic stethoscope which is connected to a third input of the acquisition card 6 in order to produce a phonocardiogram whose results will be processed and analyzed by the software of the computer 7.

It should moreover be understood that the foregoing description has been given only by way of example and that it in no way limits the field of the invention which would not be departed from if the described details of embodiments were replaced by any other equivalent.

1. A method of acquiring electric signals from a human patient that permits suppression of electromagnetic disturbances, comprising the steps of:
providing an acquisition card that sends signals from a human patient to an electrocardiograph;
connecting a first connection element to the acquisition card, the first connection element having plural first electrodes and a first cable that includes plural first wires that are each connected to a respective different one of the first electrodes and that are parallel to each other between the respective first electrodes and the acquisition card;
connecting a second connection element to the acquisition card, the second connection element having plural second electrodes and a second cable that includes plural second wires that are each connected to a respective different one of the second electrodes and that are parallel to each other between the respective second electrodes and the acquisition card;
attaching the first connection element to the human patient, a first set of at least two of the first electrodes being attached to a right side of the human patient relative to a sternum of the human patient and a second set of at least two of the first electrodes being attached to a left side of the human patient relative to the sternum, wherein all of the first electrodes of one of the first and second sets are arranged consecutively on a first portion of the first cable that does not cross the sternum; and
attaching the second connection element to the human patient, one of the second electrodes being attached below the heart of the human patient and a third set of at least two of the second electrodes being attached to the human patient along a line extending downward away from the heart,
whereby electromagnetic disturbances are suppressed.

2. The method of claim 1, further comprising the step of mounting each of the first and second electrodes on a respective separate support that is separately positioned on the human patient.

3. The method of claim 2, wherein one of the first and second sets of the first electrodes, other than the set arranged consecutively on the first portion of the first cable, includes ones of the first electrodes that are separated from each other along the first cable by the first portion.

4. The method of claim 1, further comprising the step of mounting the first set of the first electrodes on a single first support that is attached to the right side of the human patient and mounting the second set of the first electrodes on a single second support that is attached to the left side of the human patient.

5. The method of claim 4, wherein one of the first and second sets of the first electrodes, other than the set arranged consecutively on the first portion of the first cable, includes ones of the first electrodes that are arranged consecutively on a second portion of the first cable that is separate from the first portion and that does not cross the sternum.

6. The method of claim 4, further comprising the step of mounting the third set of the second electrodes on a single third support that is attached to the human patient along the line extending downward to the right side of the human patient.
